(19) United States
(12) Patent Application Publication
(10) Pub. No.: US 2019/0365707 A1
(43) Pub. Date: Dec. 5, 2019

(54) METHODS OF AMELIORATING POST-DIALYSIS WASHOUT AND NUTRITIONAL SUPPLEMENTS FOR USE IN SUCH METHODS

(71) Applicant: CALWOOD NUTRITIONALS, LLC, Sykesville, MD (US)

(72) Inventor: Gary J. CALTON, Sykesville, MD (US)

(73) Assignee: CALWOOD NUTRITIONALS, LLC, Sykesville, MD (US)

(21) Appl. No.: 16/509,653

(22) Filed: Jul. 12, 2019

Related U.S. Application Data
(63) Continuation of application No. 14/692,151, filed on Apr. 21, 2015, now abandoned.

(51) Int. Cl. A61K 31/405; A61K 31/4172; A61K 31/198

(52) U.S. Cl. CPC A61K 31/405; A61K 31/198; A61K 31/4172

(57) ABSTRACT

Methods of ameliorating, treating, or reducing post-dialysis washout or hangover comprising administering, near the end of or subsequent to a dialysis session, one or more or a group of amino acids and/or their hydroxy or keto analogs. The group of amino acids may be one or more members selected from the group consisting of histidine, alanine, isoleucine, arginine, leucine, asparagine, lysine, aspartic acid, methionine, cysteine, phenylalanine, glutamic acid, threonine, glutamine, tryptophan, glycine, valine, pyrrolysine, proline, selenocysteine, serine, and tyrosine, or their keto or hydroxy analogs and mixtures thereof.

### CROSS REFERENCE TO RELATED APPLICATIONS

This application is a continuation of U.S. application Ser. No. 14/692,151, filed Apr. 21, 2015, the contents of which are hereby incorporated by reference as though set forth in full herein.

### BACKGROUND OF THE INVENTION

### 1. Field of the Invention

The present invention relates to methods of ameliorating or reducing post-dialysis washout (also known as post-dialysis hangover) in a patient subjected to dialysis. Also provided are nutritional supplements for use in such amelioration or treatment.

### 2. Description of Related Art

Following dialysis, especially hemodialysis, patients may experience a syndrome or period of time called “washout.” Washout refers to a spectrum of symptoms which manifests after dialysis and thus, following dialysis the patient feels weak, tremulous, and extreme fatigue. One or more, and frequently all of these symptoms may be present to varying degrees. Patients report they are too tired, too weak to converse, hold a book or even a newspaper. Symptoms may also vary in intensity ranging from whole body aching, stiffness in joints and other flu-like symptoms including headaches, nausea and loss of appetite. The syndrome may begin toward the end of treatment or minutes following the treatment. It may last 30 minutes or 12-14 hours in a dissipating form. Patients, although exhausted, have difficulty falling asleep. Eating a light meal, rest and providing a quiet environment may help the patient cope with washout until it has “worn away.” “Step-by-step Description of Hemodialysis”, (Wikipedia, http://en.wikipedia.org/wiki/Step-by-step description of hemodialysis, Feb. 8, 2015; Apr. 8, 2015).

There have been attempts to overcome this washout period by various routines. One reported attempt was daily dialysis. In 1975 another attempt to implement daily, short dialysis was made at Maimonides Hospital in Brooklyn, N.Y. In 11 patients, many beneficial effects were noted. Most significantly, the patients reported disappearance of all uremic symptoms and post-dialysis ‘washout’ and fatigue. (Manohar N L, Louis B M, Gorfien P, Lipner H I, Success of Frequent Short Hemodialysis, Trans Am Soc Artif Intern Organs1981; 27: 604-609 as cited in Daily Dialysis: is this a Reasonable Option for the New Millennium? Twardowski, Z, J. Nephrol. Dial Transplant. 2001 July;16(7):132) This option is far too expensive to be feasible, and it is extremely limiting for the lifestyle of the patient.

One theory is that such side effects are caused by removing too much fluid and/or removing fluid too rapidly and that this results in low blood pressure, fatigue, chest pains, leg-cramps, nausea and headaches, etc. These symptoms can occur during the treatment, and can persist, post treatment; they are sometimes collectively referred to as the dialysis hangover or dialysis washout. The severity of these symptoms is usually proportionate to the amount and speed of fluid removal. However, the impact of a given amount or rate of fluid removal can vary greatly from person to person, day to day and session to session. These side effects can be avoided and/or their severity lessened by limiting fluid intake between treatments or increasing the dose of dialysis e.g. dialyzing more often or longer per treatment than the standard three times a week, 3-4 hours per treatment schedule. (http://www.wikiwand.com/en/Hemodialysis, Mar. 7, 2015.)

Still other investigators have attributed a portion of the post-dialysis washout syndrome to lack of vitamins after dialysis. (Washout of Water-soluble Vitamins and of Homocysteine During Haemodialysis: Effect of High-flux and Low-flux Dialyser Membranes, Heinz J, Domröse U, Westphal S, Luley C, Neumann KH, Dierkes J. Nephrology (Carlton). 2008 October;13(5):384-9.)

It has long been known that the oral essential amino acids (EAA) and mixtures of the essential amino acids with their keto analog salts, sodium or calcium phenylpyruvate, sodium or calcium alpha-keto isovalerate, sodium or calcium alpha-keto-isocaproate, sodium or calcium alpha-keto-methylvalerate, as disclosed in U.S. Pat. No. 4,100,161, are valuable for raising the serum albumin when taken as a supplement to a dialysis patient's normal diet. U.S. Pat. No. 4,100,161 is hereby incorporated by reference in its entirety, as though set forth in full herein. Serum albumin levels are the best indicator of the morbidity and mortality on the dialysis patient in succeeding years on dialysis. However, none of the investigators studying the effects of amino acids has utilized or suggested the use of an amino acid formula for alleviating post-dialysis washout, which is the most prominent side effect of dialysis.

Previous workers in this area, although aware of the problem, did not realize that the use of one or more or a group of amino acids could counter the effect of post-dialysis washout, even though it was known that the circulating level of amino acids in the body dropped precipitously when the patient was dialyzed. Walser has shown that by taking the EAA, kidney patients on dialysis could improve their serum albumin, the best predictor of survival on dialysis. (Randomized Double-blind Trial of Oral Essential Amino Acids for Dialysis-associated Hypoalbuminemia, Eustace J A, Coresh J, Kutchey C, Te P L, Gimenez L F, Scheel P J, Walser M. Kidney Int. 2000 June; 57(6):2527-38.) Thus, it was not understood or studied that the timely administration of amino acids could result in the amelioration or reduction of symptoms of post-dialysis syndrome.

It was previously known that the essential amino acids promoted protein synthesis and that the non-essential amino acids did not stimulate muscle synthesis. (Smith K., Reynolds N., Downie S., Patel A., Rennie M. J.(1998) Effects of Flooding Amino Acids on Incorporation of Labeled Amino Acids into Human Muscle Protein. Am. J. Physiol. Endocrinol. Metab. 275:E73 http://ajpendo.physiology.org/content/275/1/E73?ijkey=5f4ad6768f69822e8d4ad161112350213bfeef43&keytype2=tf_ipsecsha.) However, the presence of the non-essential amino acids did not prevent the muscle synthesis by the essential amino acids. (Tipton K. D., Ferrando A. A., Phillips S. M., Doyle D., Wolfe R. R. (1999), Postexercise Net Protein Synthesis in Human Muscle from Orally Administered Amino Acids, Am. J. Physiol. Endocrinol. Metab. 276:E628-E634. http://ajpendo.physiology.org/content/276/4/E628?ijkey=0c27e89d7dbdd646e94b5418a636ec2e4cf9e35d&keytype2=tf_ipsecsha)

It has been shown that the increase in arterial amino acid concentrations during exercise was approximately 100% when ingesting EAA. (Tipton K D, Rasmussen B B, Miller S L, Wolf S E, Owens-Stovall S K, Petrini B E, Wolfe R R, Timing of Amino Acid-carbohydrate Ingestion Alters Anabolic Response of Muscle to Resistance Exercise Am J Physiol Endocrinol Metab 281: E197-E206, 2001 http://ajpendo.physiology.org/content/281/2/E197?ijkey=086ee3482672b192f55ee47e220d2392 8a763d47&keytype2=tf_ipsecsha) but only approximately 30% for whey proteins after ingestion. (Tipton K D, Elliott T A, Ferrando A A, Aarsland A A, Wolfe R R, Stimulation of Net Muscle Protein Synthesis by Whey Protein Ingestion Before and After Exercise. Am J Physiol Endocrinol Metab 2007 January;292(1):E71-6 http://ajpendo.physiogy.org/content/292/1/E71.long)

It is known that the amino acids are dialysable, and that their level in the bloodstream is greatly reduced after dialysis. To prevent this, research has been carried out on maintaining the level of amino acids in the fluids by adding the amino acids to the fluid used in dialysis. (Amino Acid-based Peritoneal Dialysis Solutions for Malnutrition: New Perspectives, Tjiong H L, Swart R, van den Berg J W, Fieren M W, Peri Dial Int. 2009 July-August;29(4):384-93, http://www.pdiconnect.com/content/29/4/384.long) While this is effective, it is also very expensive due to the costs of providing a sterile solution.

Other formulations of EAA are effective in renal patients and might be used. Furst et al. designed a new formula of essential amino acids, with a higher proportion of valine, lower proportions of leucine and isoleucine, and the inclusion of tyrosine, in an attempt to correct the extracellular and intracellular abnormalities of amino acid concentration that they found in predialysis uremic patients. They have shown that this mixture maintains nitrogen balance while improving abnormalities of amino acid concentrations. This formula was also used in the Feasibility Phase of a large NIH-supported study entitled “Modification of Diet in Renal Disease”, and was found to maintain nutrition in these patients with advanced chronic renal failure. This mixture has neither been used nor advocated in patients for treating dialysis syndrome. (Furst P, Alvestrand A, Bergstrom J. Effects of Nutrition and Catabolic Stress on Intracellular Amino Acid Pools in Uremia. Am J Clin Nutr 33: 1387-95, 1980; Alvestrand A, Furst P. Bergstrom J., Plasma and Muscle Amino Acids in Uremia: Influence of Nutrition with Amino Acids. Clin Nephrol 18: 297-305, 1982; Alvestrand A, Ahlberg M, Furst P, Bergstrom J., Clinical Results with a Low Protein Diet and a New Amino Acid Preparation in Patients with Chronic Uremia, Clin Nephrol 19: 67-73, 1983; Bergstrom J, Ahlberg M, Alvestrand A, Furst P., Amino Acid Therapy for Patients with Chronic Renal Failure, Infusionsther Klin Ernahr; Modification of Diet in Renal Disease Study Group. The Modification of Diet in Renal Disease Study: Design, Methods, and Results from the Feasibility Study. Am J Kidney Dis 20: 18-33, 1992.

It is known that a solution of EAA moved out of an empty stomach within 25 minutes and that over the next 45 minutes, the level of circulating EAA in the bloodstream was elevated. After one pass of blood through the body, the EAA were completely absorbed by the organs and used for muscle and tissue formation. This higher level of EAA in the bloodstream returned to normal within 45 minutes after consumption on an empty stomach, with no formation of creatinine or blood urea nitrogen. (Rasmussen B B, Tipton K D, Miller S L, Wolf S E, Wolfe R R, An Oral Essential Amino Acid-carbohydrate Supplement Enhances Muscle Protein Anabolism after Resistance Exercise, J Appl Physiol. 2000 February; 88(2):386-92. Accessed at http://jap.physiology.org/content/88/2/386.) The minimization of creatinine and blood urea nitrogen, removed by dialysis, is especially important to the kidney patient.

In addition, whey ingestion results in significant urea formation but the essential amino acids do not produce urea or creatinine in the body upon ingestion. (Rasmussen B B, Tipton K D, Miller S L, Wolf S E, Wolfe R R. An oral essential amino acid-carbohydrate supplement enhances muscle protein anabolism after resistance exercise. J Appl Physiol. 2000 Febraury;88(2):386-92. http://jap.physiology.org/content/88/2/386.)

However, post-dialysis washout, especially fatigue, remains a significant problem in dialysis patients. In fact, fatigue remains a very important factor to patients, as illustrated by a recent survey which demonstrated that 94% of surveyed dialysis patients would accept more frequent hemodialysis if it would increase their energy, whereas only 19% would exchange more frequent treatments for a three-year increase in survival. (Correlates and Outcomes of Fatigue among Incident Dialysis Patients, Jhamb et al., Clinical Journal of the American Society of Nephrology, 1779-1786, 2009.)

Thus, prior to the present invention, no feasible treatment for overcoming (i.e., ameliorating, reducing or treating) these side effects of dialysis has been available and virtually every patient on hemodialysis experiences these side effects, at least to some degree. Post dialysis syndrome impacts virtually every dialysis patient's quality of life. Thus, there has been a continuing need for a way of ameliorating, reducing or treating these side effects and symptoms.

### SUMMARY OF THE INVENTION

It has now been now found that the administration of one or more amino acids, or a group of amino acids, can markedly improve the post-dialysis washout symptoms suffered by patients undergoing dialysis, including hemodialysis or peritoneal dialysis.

The invention provides methods for ameliorating, reducing or treating post-dialysis washout in a patient subjected to dialysis, comprising administering to the patient, near the end of, or subsequent to a dialysis session, an effective amount of at least one amino acid. The method may comprise administering a group of at least two amino acids, which may be selected from two or more of histidine, alanine, isoleucine, arginine, leucine, asparagine, lysine, aspartic acid, methionine, cysteine, phenylalanine, glutamic acid, threonine, glutamine, tryptophan, glycine, valine, pyrrolysine, proline, selenocysteine, serine, and tyrosine, and/or their hydroxy or keto analogs. The group of amino acids may be selected from two or more of histidine, isoleucine, leucine, lysine, methionine, phenylalanine, threonine, tryptophan, and valine and/or their hydroxy or keto analogs. The group of amino acids may be selected from two or more of isoleucine, leucine, and valine and/or their hydroxy or keto analogs.

The method may comprise administering the one or more, or group of amino acids within a period of from about 45 minutes before the end of a dialysis session to about 2 hours from the end of the dialysis session. The one or more or group of amino acids may be administered within a period of from about 30 minutes before said dialysis to about 1 hour from the end of the dialysis session. The method may comprise administering the one or more or a group of amino acids within a period of from about 20 minutes before the end of a dialysis session to about 30 minutes after the end of the dialysis session. The method may comprise administering one or more or a group of amino acids within a period of from about 10 minutes before the end of the dialysis session to about 10 minutes after the end of the dialysis session. The one or more or group of amino acids may be administered in multiple doses.

Also provided are methods of ameliorating, reducing or treating post-dialysis washout in a patient subjected to dialysis, comprising administering, prior to the end of and/or subsequent to a dialysis session, an effective amount of a nutritional supplement comprising a group of amino acids. The group of amino acids may comprise at least two amino acids selected from the group consisting of histidine, alanine, isoleucine, arginine, leucine, asparagine, lysine, aspartic acid, methionine, cysteine, phenylalanine, glutamic acid, threonine, glutamine, tryptophan, glycine, valine, pyrrolysine, proline, selenocysteine, serine, and tyrosine, and mixtures thereof and/or their hydroxy or keto analogs. The group of amino acids may comprise at least two amino acids selected from the group consisting of histidine, isoleucine, leucine, lysine, methionine, phenylalanine, threonine, tryptophan, and valine, and mixtures thereof and/or their hydroxy or keto analogs. The group of amino acids may comprise at least two amino acids selected from the group consisting of isoleucine, leucine, and valine and mixtures thereof and/or their hydroxy or keto analogs.

The nutritional supplement may be administered within a period of from about 10 minutes to about 2 hours of end of the dialysis session.

The nutritional supplement may further comprise a sweetener. The sweetener may be selected from the group consisting of a sugar, a non-nutritive sweetener, and mixtures thereof. The sweetener may be selected from the group consisting of aspartame, acesulfam-K, cyclamate, saccharin, sucralose, sucrose, fructose, galactose, maltose, lactose, brazzein, Curculin, erythritol, glycerol, glycyrrhizin, hydrogenated starch hydrolysates, inulin, isomalt, lactitol, mabinlin, maltitol, malto-oligosaccharide, mannitol, miraculin, extracts of S. grosvenorii fruit, monatin, monellin, osladin, pentadin, sorbitol, stevia and extracts thereof, tagatose, thaumatin and xylitol, or mixtures thereof.

The nutritional supplement may further comprise a component selected from the group consisting of at least one non-nutritive fiber, at least one protein, at least one oil, at least one vitamin, at least one peptide, at least one carbohydrate, at least one mineral, at least one natural or artificial flavor, and mixtures thereof.

Also provided are methods of ameliorating post-dialysis washout comprising administering, near the end of or subsequent to a dialysis session, an effective amount of a nutritional supplement comprising:
A) a group of amino acids selected from the group consisting of histidine, alanine, isoleucine, arginine, leucine, asparagine, lysine, aspartic acid, methionine, cysteine, phenylalanine, glutamic acid, threonine, glutamine, tryptophan, glycine, valine, pyrrolysine, proline, selenocysteine, serine, and tyrosine and mixtures thereof and/or their hydroxy or keto analogs;
B) at least one sweetener selected from the group consisting of a sugar, a non-nutritive sweetener or a mixture thereof; and
C) at least one component selected from the group consisting of at least one non-nutritive fiber, at least one protein, at least one oil, at least one vitamin, at least one peptide, at least one carbohydrate, at least one mineral, at least one natural or artificial flavor, and mixtures thereof.

### DETAILED DESCRIPTION OF THE INVENTION

As used herein, the singular forms “a,” “an,” and “the” include the plural reference unless the context clearly dictates otherwise. For example, reference to “an amino acid” would also mean that mixtures of one or more amino acids, or a group of amino acids, can be present unless specifically excluded. As used herein, “one or more”, “two or more” and “group of amino acids” may be used interchangeably and are also intended to include their hydroxy and/or keto analogs.

Except where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and claims are approximations that may vary depending upon the desired properties sought to be obtained by the present invention. At the very least, and not to be considered as an attempt to limit the application of the doctrine of equivalents to the scope of the claims, each numerical parameter should be construed in light of the number of significant digits and ordinary rounding conventions.

Additionally, the recitation of numerical ranges within this specification is considered to be a disclosure of all numerical values and ranges within that range. For example, if a range is from about 1 to about 50, it is deemed to include, for example, 1, 7, 34, 46.1, 23.7, or any other value or range within the range.

Unless otherwise stated, a reference to a compound or component includes the compound or component by itself, as well as in combination with other compounds or components, such as mixtures of compounds.

The various embodiments disclosed herein can be used separately and in various combinations unless specifically stated to the contrary.

In accordance with the present invention, the administration of one or more, or a group of amino acids, can markedly improve the post-dialysis washout symptoms suffered by patients undergoing hemodialysis and various other forms of dialysis, such as peritoneal dialysis where the “washout” is far less pronounced than in hemodialyis.

The correct timing of taking a group of the amino acids is very important. Allowing time for the amino acid mixture to move through the stomach is required as dosage too early will result in lost amino acids due to dialysis. However, if the amino acids are timed to be going out of the stomach near the end of dialysis, they will immediately be available to increase circulating levels of amino acids in the blood stream, removing the body's need to break down muscle to obtain the correct level of circulating amino acids in the blood stream. This also avoids the long wait time for protein breakdown from a meal.

It has been found that during a period of from approximately 45 minutes prior to the end of hemodialysis to 2 hours after the end of hemodialysis, the drinking of a solution or eating a matrix of amino acids ameliorates post-dialysis hangover. The one or more, or group of amino acids may administered or fed to a patient in the form of a liquid drink or solid matrix or can be in the form of capsules, pills, pastes or jellies. Suitable solid matrix formulations are disclosed in U.S. Pat. No. 7,223,417. U.S. Pat. No. 7,223,417 is hereby incorporated by reference in its entirety, as though set forth in full herein. It has further been found that if the group of amino acids are contained in a matrix such as a soluble dietary fiber, e.g., as disclosed in U.S. Pat. No. 7,223,417, a period of 20-40 minutes prior to the end of dialysis is best for amelioration of the post-dialysis syndrome since it allows time for the amino acids to be leached from the matrix and pass out of the stomach and into the intestines, where they are then picked up by the blood supply and circulated through the body to the organs over the following 45 minutes. Such a matrix is especially valuable in administering the group of amino acids without adding fluid for those dialysis patients on a liquid restriction.

Without wishing to be bound by theory, it is speculated that a correction of the low circulating amino acid levels known to occur in dialysis is achieved by the ingestion of amino acids at the appropriate time, thus reducing the extraction of the amino acids in the patient's muscle to achieve the body's desired level of circulating amino acids. It is further speculated (without wishing to be bound by theory) that the amelioration of the dialysis patient's washout is achieved by this correction.

It is desirable that the group of amino acids be incorporated in a palatable formula. Examples of such formulae include the drink mix and solid matrix forms discussed below. The essential amino acids are a preferred supplement, especially in a balanced formula; however, substantial variation of the amounts and percentages of individual essential amino acids may be used. To a greater or lesser degree, the branched chain amino acids may be used to initiate amelioration of post-dialysis “washout” since they are known to reduce muscle degradation. (Effect of BCAA Intake During Endurance Exercises on Fatigue Substances, Muscle Damage Substances, and Energy Metabolism Substances. Kim D H, Kim S H, Jeong W S, Lee H Y. J Exerc Nutrition Biochem. 2013 December;17(4):169-80; this document is hereby incorporated herein by reference as though set forth in full herein). The addition of non-essential amino acids will also be of value since some are known to be useful in stimulating muscle synthesis, e.g. creatine. (The Effects of Pre Versus Post Workout Supplementation of Creatine Monohydrate on Body Composition and Strength, Antonio J, Ciccone V. J Int Soc Sports Nutr. 2013 Aug. 6;10:36; this document is hereby incorporated herein by reference as though set forth in full herein.)

Suitable solutions for use in the invention are exemplified by NUTRASENTIALS drink mix, available from Calwood Nutritionals. Suitable solid matrix formulations are exemplified by NUTRAMINE AMINOBITES, available from Calwood Nutritionals. Another suitable matrix form is that of a pill of the essential amino acids, such as that used by Walser in U.S. Pat. No 6,713,501. U.S. Pat. No. 6,713,501 is hereby incorporated by reference as though set forth in full herein.

As stated above, the supplement for amelioration of post-dialysis syndrome may include one or more or a group of amino acids selected from the group comprising histidine, alanine, isoleucine, arginine, leucine, asparagine, lysine, aspartic acid, methionine, cysteine, phenylalanine, glutamic acid, threonine, glutamine, tryptophan, glycine, valine, pyrrolysine, proline, selenocysteine, serine, and tyrosine. Preferably, all the amino acids may be in their L-form, however, amounts of D-amino acids sometimes accompany the L-amino acids. A further valued group of amino acids to be utilized for the alleviation of the post-dialysis syndrome comprises histidine, isoleucine, leucine, lysine, methionine, phenylalanine, threonine, tryptophan, and valine. A still further valued group of amino acids to be utilized for the relief of the post-dialysis syndrome comprises isoleucine, leucine, and valine.

Suitable groups of amino acids are exemplified by NUTRAMINE products, also available from Calwood Nutritionals in the form of USP grade amino acid nutritional products.

Such products are exemplified by the following characteristics:

TABLE 1
Nutramine USP Grade Amino Acids

| | | |
|---|---|---|
| L-Histidine | 8.0% | 280 mg |
| L-Isoleucine | 10.1% | 360 mg |
| L-Leucine | 15.9% | 560 mg |
| L-Lysine | 11.6% | 410 mg |
| L-Methionine | 15.9% | 560 mg |
| L-Phenylalanine | 15.9% | 560 mg |
| L-Threonine | 7.3% | 250 mg |
| L-Tryptophan | 3.6% | 130 mg |
| L-Valine | 11.6% | 410 mg |
| TOTAL | 100.0% | 3520 mg |

% AS A TOTAL OF AMINO ACIDS PRESENT mg PER SERVING VALUE

Suitable groups of amino acids are also exemplified by AMINOBITES products (a solid matrix form for administration), also available from Calwood Nutritionals, which have the following exemplified characteristics:

TABLE 2
AminoBites USP Grade Amino Acids

| | | |
|---|---|---|
| L-Histidine | 6.2% | 220 mg |
| L-Isoleucine | 8.3% | 290 mg |
| L-Leucine | 12.5% | 440 mg |
| L-Lysine | 9.0% | 310 mg |
| L-Methionine | 12.5% | 440 mg |
| L-Phenylalanine | 9.7% | 340 mg |
| L-Threonine | 9.0% | 320 mg |
| L-Tryptophan | 3.5% | 120 mg |
| L-Tyrosine | 10.4% | 360 mg |
| L-Valine | 18.8% | 660 mg |
| TOTAL | 100.0% | 3500 mg |

% AS A TOTAL OF AMINO ACIDS PRESENT and mg PER SERVING

In the case of NUTRASENTIALS drink mix, the drink mix is is added to water or juice. Add the Mix to a 12-16 ounce drink bottle containing 8 ounces of water or juice and then shake vigorously. One may heat, refrigerate, add ice or freeze (a frozen ice dessert), depending on preference.

In some preferred formulations, arginine is added in an amount of: arginine from about 1 mg/serving to about 5000 mg/serving, more preferably about 100 mg to about 3000 mg/serving, still more preferably about 250 mg/serving to about 1000 mg/serving.

In some most preferred embodiments, arginine is added in amount of about 12.5% by weight, such as by adding arginine to the above NUTRAMINE and/or NUTRAMINE AMINOBITES formulations in such amounts.

Suitable formulations include the following:

TABLE 3

| | Exemplified | | Exemplified Ranges | |
|---|---|---|---|---|
| | Formula | | From about | To about |
| | mg | | mg/serving | mg/serving |
| phenylalanine | 560 | 15.9% | 131 | 858 |
| leucine | 560 | 15.9% | 132 | 840 |
| methionine | 560 | 15.9% | 132 | 819 |
| tryptophan | 130 | 3.7% | 31 | 249 |
| isoleucine | 360 | 10.2% | 80 | 560 |
| lysine | 410 | 11.6% | 99 | 629 |
| threonine | 250 | 7.1% | 58 | 400 |
| valine | 410 | 11.6% | 96 | 607 |
| histidine | 280 | 8.0% | 66 | 426 |
| | 3520 | | | |

TABLE 4

| | Exemplified | | Exemplified Ranges | |
|---|---|---|---|---|
| | formula | | From about | To about |
| | mg | | mg/serving | mg/serving |
| phenylalanine | 560 | 15.9% | 378 | 681 |
| leucine | 560 | 15.9% | 400 | 464 |
| methionine | 560 | 15.9% | 387 | 683 |
| tryptophan | 130 | 3.7% | 93 | 156 |
| isoleucine | 360 | 10.2% | 240 | 440 |
| lysine | 410 | 11.6% | 292 | 673 |
| threonine | 250 | 7.1% | 164 | 309 |
| valine | 410 | 11.6% | 286 | 500 |
| histidine | 280 | 8.0% | 189 | 355 |
| | 3520 | | | |

In the formulae herein, including the above tables, all parts and percentages are by weight.

Essential amino acids, as above, and mixtures of the essential amino acids with their keto analog salts, sodium or calcium phenylpyruvate, sodium or calcium alpha-keto isovalerate, sodium or calcium alpha-keto-isocaproate, sodium or calcium alpha-keto-methylvalerate, as disclosed in U.S. Pat. No. 4,100,161 (incorporated by reference above), may also be employed.

One of ordinary skill in the art will readily know how to formulate suitable drink and solid matrix formulations based on the foregoing.

In general, the active ingredient in the formulations of the invention which are to be fed or administered to a patient comprise the one or more amino acids discussed herein. The total amount of the active ingredient to be administered will generally range from about 2 grams to about 10 grams, preferably from about 2.5 grams to about 9 grams, and more preferably from about 3.5 grams to about 8 grams per serving.

Thus, a unit dosage or feeding may contain the foregoing amounts of active ingredient, and can be administered one or more times per post-dialysis feeding.

Preferably, multiple feedings are administered, such as one feeding before the end of dialysis and one shortly after the end of dialysis. In the case where 2 such feedings are administered, each feeding would be in an amount of from about 1.5 grams to 4.5 grams, preferably from about 2 to about 4 grams and more preferably about 3.5 grams of active ingredient (one or more amino acids).

The rapid introduction to the bloodstream of the ingested amino acids is not matched by protein supplements based on milk proteins, whey and casein, which are used as a very cheap protein source. Milk proteins (casein and whey) take longer than the EAA to raise the circulating level of amino acids. However, use of these products in varying amounts to reduce the osmotic load of the amino acids may be useful in some subjects. Whey is much more rapidly taken up by the body than casein. Protein synthesis was stimulated by 68% with a whey meal and to a lesser extent (+31%) with the casein meal. (Boirie Y, Dangin M, Gachon P, Vasson M P, Maubois J L, Beaufrere B, Slow and Fast Dietary Proteins Differently Modulate Postprandial Protein Accretion, Proc Natl Acad Sci USA. 1997 Dec. 23;94(26):14930-5.) Both whey and casein are common components of foods like ENSURE (listed as milk protein concentrate on the bottle) or NEPRO (Milk Protein Isolate). Although proteins and non-essential amino acids may be present in the mixture of this invention, they are not thought to be as important since the non-essential amino acids do not stimulate muscle synthesis and protein supplements will enter into the bloodstream at a slower rate, diminishing their value in preventing post-dialysis syndrome.

Other components of the supplement additionally comprise a sweetener. The sweetener may be selected from those sweeteners consisting of a sugar, a non-nutritive sweetener or a mixture thereof. Said sweetener may be taken from the group consisting of aspartame, acesulfam-K, cyclamate, saccharin, sucralose, sucrose, fructose, galactose, maltose, lactose, brazzein, Curculin, erythritol, glycerol, glycyrrhizin, hydrogenated starch hydrolysates, inulin, isomalt, lactitol, mabinlin, maltitol, malto-oligosaccharide, mannitol, miraculin, extracts of S. grosvenorii fruit, monatin, monellin, osladin, pentadin, sorbitol, stevia and extracts thereof, tagatose, thaumatin and xylitol, or mixtures thereof.

Additionally, the supplement may contain a either soluble or insoluble fiber. Especially valuable fibers include carrageenans, starch polysaccharides such as arabinoxylans, cellulose, and other plant components such as resistant starch, resistant dextrins, inulin, lignin, waxes, chitins, pectins, beta-glucans, and oligosaccharides as well as gum fibers such as guar gum and acacia gum.

Amounts of protein, e.g., whey or casein, or protein hydrolysates may also be included in addition to oil, vitamins, peptides, carbohydrates, natural and/or artificial flavors and minerals.

The method for amelioration of dialysis “hangover,” may comprise administration of the amino acids shortly before the end to shortly after the end of the dialysis. Thus, advantageously, the nutritional supplements used herein are administered or fed to the patient during the period of from about 45 minutes prior to the end of dialysis to about 2 hours after dialysis, including from about 30, 20, 10 and/or 0 minutes prior to the end of dialysis to about 1 hour, 30, 15, 10 and/or 5 minutes after dialysis.

In hemodialysis, “end of dialysis” is understood to occur at the time the patient is disconnected from the dialysis membrane or filter, typically when blood lines are removed.

The same is true for the administration of the amino acids shortly after or just before changing bags in peritoneal dialysis. Ideally, the administration of said supplement in single or multiple doses and in varying dose quantities, would be within 1-2 hours of changing the bag.

Multiple doses or feedings in liquid or solid matrix form may advantageously be administered. In such cases, one or more feedings or doses may be administered at any time during the above-given time periods. There is no limit on the number of separate doses that may be administered and 2, 3, 4, 5 or more doses or feedings may be administered.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

### EXAMPLES

### Example 1

A hemodialysis patient took a mixture of a balanced formula of essential amino acids (EAA) such as those found in NUTRASENTIALS Drink Mix after dialysis sessions over a period of weeks. She was advised to test the drink by the administration of the EAA 30, 20, 10 and 0 minutes prior to the end of succeeding dialysis sessions and 10, 20, 30 and 45 minutes after the end of her dialysis session. All the timed intakes improved the symptoms of her post-dialysis syndrome, however, the greatest reduction in her “washout” symptoms was achieved by taking the drink 10 minutes prior to the end of her dialysis session.

She repeated the process using a balanced formula of essential amino acids (EAA) such as those found in Nutramine AminoBites, prepared as described in U.S. Pat. No. 7,223,417, at the same time intervals and found that she obtained maximal reduction of the washout symptoms by taking the EAA in this formulation 25 minutes before the end of dialysis.

### Example 2

A hemodialysis patient took a mixture of a balanced formula of essential amino acids (EAA) such as those found in NUTRASENTIALS Drink Mix, 10 minutes before the end of dialysis and in a second iteration, 10 minutes before and 25 minutes after the end of dialysis. The patient felt the first dose of EAA removed the tired, weary feeling in post-dialysis hangover and that addition of the second dose was so invigorating that he could return to work.

### Example 3

A hemodialysis patient took a mixture of a balanced formula of essential amino acids (EAA) such as those found in NUTRAMINE AMINOBITES, prepared as described in U.S. Pat. No. 7,223,417, 25 minutes before the end of dialysis and in a second iteration, 25 minutes before and 25 minutes after the end of dialysis. The patient, who had been on dialysis for about one year and had suffered severely from post-dialysis syndrome, having to sleep for 2-4 hours after each dialysis session, felt very well after the first instance and found that he could exercise immediately after the second instance. The patient described himself as vastly changed in his lifestyle and having significantly greater mental acuity, better rest and greater physical stamina due to taking the product at these time periods.

The above Examples demonstrate that the correct timing of eating or drinking the amino acid(s) is important. Without wishing to be bound by theory, it is speculated that the dose of amino acids should not be given too early or some of the amino acids will be lost due to dialysis. In the same speculation, it is theorized that the amino acids should be ingested at the earliest possible time in order to amelioriate the post-dialysis washout which is reported to occur “toward the end of treatment or minutes following the treatment [and] may last from 30 minutes to 12-14 hours.” (http://en.wikipedia.org/wiki/Step-by-step_description_of_hemodialysis_accessed 2/8/15, supra.) When a fluid restriction is placed on the dialysis patient, a form of taking the amino acids which involves no liquid intake would be preferred, such as that found in Nutramine AminoBites, available from Calwood Nutritionals.

The above examples also demonstrate that the methods of the invention are effective in ameliorating and/or reducing post-dialysis washout.

### Example 4

A subject who did not have kidney disease was given the amino acids in liquid form (as Nutrasentials Drink mix) experienced “osmotic dumping” resulting in nausea within seconds of the ingestion. When given the AminoBites, the subject experienced nausea approximately 10 minutes after ingestion of the amino acids. When small quantities of food, such as crackers, were taken concomitantly with the amino acids in liquid form in small amounts, the amount of nausea due to “osmotic dumping” was reduced.

### Example 5

A kidney patient who was on peritoneal dialysis, with changes 3 times per day, 1 liter per exchange, 1 hour dialysis per exchange, took one serving of EAA in liquid form (3.5 g of EAA in 8 ounces of water) immediately after removing fluid from the peritoneum. He said this regimen allowed him to return to work and that he felt the best he had in two years.

The preceding examples can be repeated with similar success by substituting the generically and specifically described components and conditions of this invention for those used in the preceding examples.

From the foregoing description, one skilled in the art can easily ascertain the essential characteristics of this invention, and without departing from the spirit and scope thereof, can make various changes and modifications of the invention to adapt it to various subjects and conditions.

What is claimed is:

1. A method of ameliorating post-dialysis washout in a patient subjected to hemodialysis or peritoneal dialysis comprising orally administering an effective amount of a composition consisting essentially at least one free amino acid and/or a hydroxy or keto analog thereof in a pharmaceutically-acceptable solution suitable for oral administration to said patient within a period of: spanning from 45 minutes prior to the end of a dialysis session to 2 hours subsequent to the end of a dialysis session, an effective amount of at least one free amino acid and/or a hydroxy or keto analog thereof.

2. The method of claim 1 comprising administering a group of at least two free amino acids and/or their hydroxy or keto analogs.

3. The method of claim 2 wherein said group of free amino acids is selected from two or more of histidine, alanine, isoleucine, arginine, leucine, asparagine, lysine, aspartic acid, methionine, cysteine, phenylalanine, glutamic acid, threonine, glutamine, tryptophan, glycine, valine, pyrrolysine, proline, selenocysteine, serine, and tyrosine and/or their hydroxy or keto analogs.

4. The method of claim 3 wherein said group of free amino acids is selected from two or more of histidine, isoleucine, leucine, lysine, methionine, phenylalanine, threonine, tryptophan, and valine and/or their hydroxy or keto analogs.

5. The method of claim 4 wherein said group of free amino acids is selected from two or more of isoleucine, leucine, and valine and/or their hydroxy or keto analogs.

6. The method of claim 1 comprising administering said free amino acid within a period of: spanning from 30 minutes before the end of said dialysis session to 1 hour after the end of said dialysis session.

7. The method of claim 6 comprising administering said free amino acid within a period of: spanning from 20 minutes before the end of said dialysis session to 30 minutes after the end of said dialysis session.

8. The method of claim 7 comprising administering said free amino acid within a period of: spanning from 10 minutes before the end of said dialysis session to 10 minutes after the end of said dialysis session.

9. The method of claim 1 comprising administering said free amino acid in multiple doses.

10. A method of ameliorating post-dialysis washout in a patient subjected to dialysis, comprising orally administering an effective amount of a nutritional supplement comprising a group of free amino acids and/or their hydroxy or keto analogs to said patient within a period of: spanning from 45 minutes prior to the end of a dialysis session to 2 hours subsequent to the end of a dialysis session, an effective amount of a nutritional supplement comprising a group of free amino acids and/or their hydroxy or keto analogs.

11. The method of claim 10 wherein said group of free amino acids comprises at least two amino acids selected from histidine, alanine, isoleucine, arginine, leucine, asparagine, lysine, aspartic acid, methionine, cysteine, phenylalanine, glutamic acid, threonine, glutamine, tryptophan, glycine, valine, pyrrolysine, proline, selenocysteine, serine, and tyrosine, and mixtures thereof and/or their hydroxy or keto analogs.

12. The method of claim 11 wherein said group of free amino acids comprises at least two amino acids selected from histidine, isoleucine, leucine, lysine, methionine, phenylalanine, threonine, tryptophan, and valine, and mixtures thereof and/or their hydroxy or keto analogs.

13. The method of claim 12 wherein said group of free amino acids comprises at least two amino acids selected from isoleucine, leucine, and valine and mixtures thereof and/or their hydroxy or keto analogs.

14. The method of claim 10 comprising administering said nutritional supplement within a period of: spanning from 10 minutes before the end of said dialysis session to 2 hours after the end of said dialysis session.

15. The method of claim 11 wherein said nutritional supplement further comprises a sweetener.

16. The method of claim 15 wherein said sweetener is selected from the group consisting of a sugar, a non-nutritive sweetener, and mixtures thereof.

17. The method of claim 16 wherein the sweetener is selected from the group consisting of aspartame, acesulfam-K, cyclamate, saccharin, sucralose, sucrose, fructose, galactose, maltose, lactose, brazzein, Curculin, erythritol, glycerol, glycyrrhizin, hydrogenated starch hydrolysates, inulin, isomalt, lactitol, mabinlin, maltitol, malto-oligosaccharide, mannitol, miraculin, extracts of S. grosvenorii fruit, monatin, monellin, osladin, pentadin, sorbitol, stevia and extracts thereof, tagatose, thaumatin and xylitol, or mixtures thereof.

18. The method of claim 10 wherein said nutritional supplement further comprises a component selected from the group consisting of at least one non-nutritive fiber, at least one protein, at least one oil, at least one vitamin, at least one peptide, at least one carbohydrate, at least one mineral, at least one artificial or natural flavor, and mixtures thereof.

19. A method of ameliorating post-dialysis washout in a patient subjected to dialysis comprising orally administering to said patient, within a period of spanning from 45 minutes prior to the end of a dialysis session to 2 hours subsequent to the end of a dialysis session, an effective amount of a nutritional supplement comprising:
A) a group of free amino acids selected from histidine, alanine, isoleucine, arginine, leucine, asparagine, lysine, aspartic acid, methionine, cysteine, phenylalanine, glutamic acid, threonine, glutamine, tryptophan, glycine, valine, pyrrolysine, proline, selenocysteine, serine, and tyrosine and mixtures thereof and/or their hydroxy or keto analogs;
B) at least one sweetener selected from the group consisting of a sugar, a non-nutritive sweetener or a mixture thereof; and
C) at least one component selected from the group consisting of at least one non-nutritive fiber, at least one protein, at least one oil, at least one vitamin, at least one peptide, at least one carbohydrate, at least one mineral, at least one artificial or natural flavor, and mixtures thereof.
